(19) United States
(12) Patent Application Publication
(10) Pub. No.: US 2022/0323541 A1
(43) Pub. Date: Oct. 13, 2022

(54) ORTHOPOXVIRUS MAJOR HISTOCOMPATIBILITY COMPLEX (MHC) CLASS-I LIKE PROTEIN (OMCP) FOR TREATMENT OF AUTOIMMUNE DISEASE

(71) Applicant: COURIER THERAPEUTICS, INC., Boston, TX (US)

(72) Inventors: Alexander Sasha KRUPNICK, Charlottesville, VA (US); Eric Reed LAZEAR, Carrboro, NC (US); Sarah HEIN, Houston, TX (US); Daniel Marvin WATKINS, Houston, TX (US)

(73) Assignee: COURIER THERAPEUTICS, INC., Houston, TX (US)

(21) Appl. No.: 17/634,764

(22) PCT Filed: Aug. 13, 2020
(86) PCT No.: PCT/US2020/046184

§ 371 Date: Feb. 11, 2022

Related U.S. Application Data
(60) Provisional application No. 62/886,000, filed on Aug. 13, 2019.

(51) Int. Cl. A61K 38/16; A61K 47/10; A61P 11/00; A61P 19/02; A61P 37/06

(52) U.S. Cl. CPC A61K 38/162; A61K 47/10; A61P 11/00; A61P 19/02; A61P 37/06

(57) ABSTRACT

Compositions and methods for treatment of autoimmune and inflammatory conditions which include Orthopox Major Histocompatibility complex (MHC) class I-like protein (OMCP) or a variant thereof.

### CROSS-REFERENCE TO RELATED APPLICATIONS

The present application claims priority to U.S. Provisional Application No. 62/886,000, filed Aug. 13, 2019, the entirety of which is incorporated herein by reference.

### SEQUENCE LISTING

The instant application contains a Sequence Listing which has been submitted electronically in ASCII format and is hereby incorporated by reference in its entirety. Said ASCII copy, created on Aug. 12, 2020, is named 18-21031-WO_SL.txt and is 4,387 bytes in size.

### INCORPORATION BY REFERENCE

PCT Application Publication Nos. WO2016/100375 and WO2017/136818 are incorporated herein by reference in their entirety.

### BACKGROUND

Orthopoxvirus Major Histocompatibility Complex (MHC) Class-I Like Protein (OMCP) acts as a competitive antagonist of the NKG2D receptor, and can block activation triggered by the receptor. Chronic inflammation conditions like intestinal inflammation, type 1 diabetes, multiple sclerosis, rheumatoid arthritis, and chronic obstructive pulmonary disease (COPD) are possible pathologies that could be a target for OMCP therapy.

### SUMMARY

The present disclosure relates to the treatment of autoimmune diseases by administration of OMCP or a variant or mutant thereof to a subject with an autoimmune or inflammatory disease.

### DETAILED DESCRIPTION

### OMCP and OMCP Variants

In some embodiments, the methods and compositions of the present disclosure are directed to the use of full length OMCP. In other embodiments, an OMCP variant may be used such as, by way of example but not limitation, a truncated or mutated OMCP that has similar binding affinity of the full length OMCP. For example, an OMCP variant may be a truncated or mutated OMCP that has a slightly lower binding affinity relative to the binding affinity of the full length OMCP. In still other embodiments, a variant is a truncated or mutated OMCP that has a slightly higher binding affinity relative to the binding affinity of the full length OMCP. OMCP specifically binds to NKG2D with a binding affinity of about 0.1 to about 5 nM. For example, OMCP specially binds to human NKG2D with a binding affinity of about 0.2 nM and mouse NKG2D with a binding affinity of about 3 nM. In a preferred embodiment, OMCP or a variant thereof binds to human NKG2D with a binding affinity of about 1000 nM to about 0.1 nM. In certain embodiments, OMCP or a variant thereof binds to human NKG2D with a binding affinity of about 100 nM to about 0.1 nM, about 10 nM to about 0.1 nM, or about 1 nM to about 0.1 nM. In other embodiments, OMCP or a variant thereof binds to human NKG2D with a binding affinity of about 1000 nM to about 1 nM, or about 1000 nM to about 10 nM, or about 1000 nM to about 100 nM. In still other embodiments, OMCP or a variant thereof binds to human NKG2D with a binding affinity of about 100 nM to about 1 nM, or about 100 nM to 10 nM. For example, OMCP or a variant thereof binds to human NKG2D with a binding affinity of about 1000 nM, about 500 nM, about 100 nM, about 50 nM, about 10 nM, about 9 nM, about 8 nM, about 7 nM, about 6 nM about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM or about 0.1 nM. In still other embodiments, the OMCP or a variant thereof binds to human NKG2D with a binding affinity of about 1000 nM to 0.01 nM.

Binding affinity can be assessed, for example, by surface plasmon resonance. By way of example, but not limitation, binding affinity can be assessed by surface plasmon resistance by measuring the binding of the OMCP or variant thereof to human NKG2D such as by using a ProteOn XPR36 (Bio-rad) instrument.

The sequence information for the full length OMCP amino acid sequence can be found using, for example, the GenBank accession number NP_619807.1 (cowpox OMCP). A skilled artisan will appreciate that homologs of OMCP may be found in other species or viruses. For example, see Lefkowitz et al, Nucleic Acids Res 2005; 33: D311-316, which is herein incorporated by reference in its entirety, which describes eighteen OMCP variants between cowpox and monkeypox virus strains. In an embodiment, OMCP is from an orthopoxvirus. In a specific embodiment, OMCP is from a cowpox virus or a monkeypox virus. In another specific embodiment, OMCP is from the Brighton Red strain of cowpoxvirus. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule encoding OMCP. Equally, BLAST protein searches may be performed with the BLASTX program to obtain amino acid sequences that are homologous to OMCP. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. Generally a homolog will have a least 80, 81, 82, 83, 84, 85, 86, 87, 88, or 89% homology. In some embodiments, the sequence may be at least 90, 91, 92, 93, 94, 95, 96, 97, 98, 99, or 100% homologous to OMCP.

A skilled artisan will appreciate that structural homologs of OMCP may be found in other species or viruses. A structural homolog may be a protein that is structurally related but the sequence is a distal homolog. For example, OMCP has low sequence identity for endogenous NKG2D ligands however it was discovered that OMCP would bind to NKG2D based on structural homology. Structural homologs can be found in other species by methods known in the art. For example, protein structure prediction may be determined by various databases, such as Phyre and Phyre2. Such databases generate reliable protein models that may be used to determine structural homologs. The main results table in Phyre2 provides confidence estimates, images and links to the three-dimensional predicted models and information derived from either Structural Classification of Proteins database (SCOP) or the Protein Data Bank (PDB) depending on the source of the detected template. For each match a link takes the user to a detailed view of the alignment between the user sequence and the sequence of known three-dimensional structure. See www.sbg.bio.ic.ac.uk/phyre2/ for more details. Generally, a structural homolog will have a least 50, 51, 52, 53, 54, 55, 56, 57, 58, or 59% confidence with OMCP. In an embodiment, a structural homolog will have a least 60, 61, 62, 63, 64, 65, 66, 67, 68, or 69% confidence with OMCP. In another embodiment, a structural homolog will have a least 70, 71, 72, 73, 74, 75, 76, 77, 78, or 79% confidence with OMCP. In still another embodiment, a structural homolog will have a least 80, 81, 82, 83, 64, 85, 86, 87, 88, or 89% confidence with OMCP. In still yet another embodiment, a structural homolog may have at least 90, 91, 92, 93, 94, 95, 96, 97, 98, 99, or 100% confidence with OMCP. The structural information for OMCP-human NKG2D may be found using the PDB ID: 4PDC.

In some embodiments, the OMCP or variant thereof comprises the sequence set forth in SEQ ID NO: 1 (HKLAFNFNLEINGSDTHSTVDVYLDDSQIITFDGKDIRPTIPFMIGDEIFLPFYKNVFSEFF SLFRRVPTSTPYEDLTYFYECDYTDNKSTFDQFYLYNGEEYTVKTQEATNKNMWLTTSE FRLKKWFDGEDCIMHLRSLVRKMEDSKRNTG). In some embodiments, the OMCP or variant thereof comprises an amino acid sequence of at least 80% identity to SEQ ID NO: 1. By way of example, but not limitation, the OMCP variant can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 1.

In some embodiments, the OMCP or variant thereof comprises the sequence set forth in SEQ ID NO: 2 (GHKLAFNFNLEINGSDTHSTVDVYLDDSQIITFDGKDIRPTIPFMIGDEIFLPFYKNVFSEF FSLFRRVPTSTPYEDLTYFYECDYTDNKSTFDQFYLYNGEEYTVKTQEATNKNMWLTTS EFRLKKWFDGEDCIMHLRSLVRKMEDSKR). In some embodiments, the OMCP or variant thereof comprises an amino acid sequence of at least 80% identity to SEQ ID NO:2. By way of example, but not limitation, the OMCP variant can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 2.

In some embodiments, the OMCP or variant thereof comprises the sequence set forth in SEQ ID NO: 3 (HKLVHYFNLKINGSDITNTADILLDNYPIMTFDGKDIYPSIAFMVGNKLFLDLYKNIFVE FFRLFRVSVSSQYEELEYYYSCDYTNNRPTIKQHYFYNGEEYTEIDRSKKATNKNSWLIT SGFRLQKWFDSEDCIIYLRSLVRRMEDSNK). In some embodiments, the OMCP or variant thereof comprises an amino acid sequence of at least 80% identity to SEQ ID NO:3. By way of example, but not limitation, the OMCP variant can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 3.

In some embodiments, the OMCP or variant thereof comprises an amino acid sequence with at least 80% homology to amino acid positions 48-67 and 110-147 of SEQ ID NO: 1, 49-68 and 111-148 of SEQ ID NO: 2, or 48-66 and 111-148 of SEQ ID NO: 3. By way of example, but not limitation, the amino acid sequence with homology to amino acid positions 48-67 and 110-147 of SEQ ID NO: 1, 49-68 and 111-148 of SEQ ID NO: 2, or 48-66 and 111-148 of SEQ ID NO: 3, can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% confidence with respect to the reference sequences. By way of further example, but not limitation, the amino acid sequence with homology to amino acid positions 48-67 and 110-147 of SEQ ID NO: 1, 49-68 and 111-148 of SEQ ID NO: 2, or 48-66 and 111-148 of SEQ ID NO: 3, can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to the reference sequences.

### PEGylated and Glycosylated OMCP

In certain embodiments, the OMCP or variant thereof can be modified for improved systemic half-life and reduced dosage frequency. In some embodiments, N-glycans may be added to OMCP. While the biological function is typically determined by the protein component, carbohydrate can play a role in molecular stability, solubility, in vivo activity, serum half-life, and immunogenicity. The sialic acid component of carbohydrate in particular, can extend the serum half-life of protein therapeutics. Accordingly, new N-linked glycosylation consensus sequences can be introduced into desirable positions in the peptide backbone to generate proteins with increased sialic acid containing carbohydrate, thereby increasing in vivo activity due to a longer serum half-life. In another embodiment, PEG can be conjugated to OMCP. Methods of conjugating PEG to a protein are standard in the art. For example, see Kolate et al, Journal of Controlled Release 2014; 192(28): 67-81, which is hereby incorporated by reference in its entirety. In some embodiments, a composition of the invention may comprise OMCP comprising PEG and/or one or more N-glycans. In some embodiments, PEG is selected from the group consisting of PEG-10K, PEG-20K and PEG-40K.

### OMCP Fused to Fc Chain

The human immunoglobulin IgG Fc chain has a serum half life of about 14 days. In certain embodiments, the OMCP or variant thereof can be fused to the N-terminus of the human IgG Fc. In other embodiments, the OMCP or variant thereof can be fused to the C-terminus of the human IgG Fc. Methods for linking the IgG Fc chain are well known in the art.

### De-Immunization

Still further, the OMCP or variant thereof of the present disclosure can be modified to remove T cell epitopes. T cell epitopes can stimulate an immunogenic reaction upon administration of a composition to a subject. Through their presentation to T cells, they activate the process of anti-drug antibody development. Preclinical screening for T cell epitopes may be performed in silico, followed by in vitro and in vivo validation. T cell epitope-mapping tools such as EpiMatrix can be highly accurate predictors of immune response. Deliberate removal of T cell epitopes may reduce immunogenicity.

### Truncated OMCP

In another embodiment, a variant can be a truncated or mutated OMCP that has binding affinity for one or more NKG2 family receptors other than NKG2D. For example, a variant can be a truncated or mutated OMCP that has binding affinity for one or more NKG2 family receptors selected from the group consisting of NKG2A, NKG2B, NKG2C, NKG2E, NKG2F and NKG2H.

### Mutated OMCP

Mutations to OMCP may be rationally selected via structure-based knowledge or mutations to OMCP may be identified via selection-based mutagenesis. In certain embodiments, mutations may be rationally selected to occur in the OMCP-NKG2D interface to either enhance or reduce binding affinity. Computational design can also be used to design OMCP mutants. A discussion of the OMCP-NKG2D interface and residues involved in the interface is included in WO 2016/100375 and WO 2017/136818, which are both incorporated herein by reference in their entirety.

Twelve OMCP residues contact eighteen NKG2D residues to form a mixture of bond types as provided in Table 1 below:

TABLE 1
NKG2D:OMCP Interface Residues

| NKG2D-A | OMCP | Bond-type |
|---|---|---|
| Lys150 | Asp132 | Salt Bridge |
| | Trp127 | H Bond |
| | Trp127 | Φ (3) |
| Ser151 | Lys126 | H Bond |
| | Trp127 | Φ (1) |
| Tyr152 | Phe122 | H Bond |
| | Phe122 | Φ (9) |
| | Lys126 | Φ (5) |
| Met184 | Thr118 | H Bond |
| | Thr119 | Φ (1) |
| | Phe122 | Φ (5) |
| Gln185 | Arg66 | Φ (1) |
| Leu191 | Phe122 | Φ (1) |
| Tyr199 | Phe122 | Φ (4) |
| Glu201 | Arg66 | Salt Bridge |
| Thr205 | Arg66 | H Bond |
| NKG2D-B | OMCP | Bond-type |
| Leu148 | Trp127 | Φ (1) |
| Ser151 | Glu131 | H Bond |
| Tyr152 | Asp132 | H Bond |
| | Glu131 | Φ (3) |
| | Met135 | Φ (5) |
| Ile182 | Ile49 | Φ (2) |
| Glu183 | Arg142 | Salt Bridge |
| Met184 | Met135 | Φ (1) |
| | Arg138 | Φ (2) |
| | Arg142 | H Bond |
| Lys186 | Arg142 | Φ (1) |
| Leu191 | Met135 | Φ (1) |
| Glu201 | Arg138 | Salt Bridge |

Φ = carbon-to-carbon hydrophobic interactions

Three residues in each NKG2D half-site are known as core binding residues because they make contacts with all known host NKG2D ligands. The core residues of NKG2D subunit A (Tyr152, Tyr199, Met184) form two hydrogen bonds and make extensive hydrophobic contacts with OMCP residues. The core residues of NKG2D subunit A contact four OMCP residues and the most critical of these is Phe122. Phe122 makes multiple hydrophobic contacts with all three NKG2D subunit A core residues, including pi-stacking with Tyr152. Phe122 also forms a backbone-to-sidechain hydrogen bond with Tyr152. Interestingly, OMCP is the first NKG2D ligand to not utilize all six NKG2D core-binding residues, with only Met184 and Tyr152 of NKG2D subunit B contacting OMCP. NKG2D subunit B Met184 and Tyr152 each make a single hydrogen bond and hydrophobic contacts with OMCP residues. Two OMCP residues, Trp127 and Asp 132, make contacts with both NKG2D protomers. OMCP Trp127 forms a hydrogen bond to Lys150 of NKG2D subunit A and makes several hydrophobic contacts with Leul48 of NKG2D subunit B. Lys150 and Ser151 of NKG2D subunit A. OMCP Asp132 forms a hydrogen bond with Tyr152 of NKG2D subunit B and a salt bridge with Lys150 of NKG2D subunit A.

Thus, it should be understood that the mutations to OMCP or the variant thereof can include mutations at the corresponding residues of OMCP identified in Table 1. It should be understood that, depending on the original sequence of OMCP, these positions may be shifted or contain different residues based on the species origin of the OMCP sequence. For example, the residues identified in Table 1 are from the Brighton Red strain of CPVX which has >60% homology to 17 other OMCP variants. In these 17 OMCP variants, 9 out of the 12 residues are identical but 3 contain conservative hydrophobic substitutions (I49L, T118I and M135I). In some embodiments, the OMCP or variant thereof includes one or mutations at the residues identified in Table 1 or corresponding residues of the sequence thereof. For example, SEQ ID NO: 1 includes Trp127 while SEQ ID NOs: 2-3 include Trp128 resulting in a shift in amino acid position of +1. Thus, any mutation intended to be at the residue corresponding to Trp127 would be made at Trp128 in SEQ ID NOs: 2-3. In some embodiments, the OMCP or variant thereof can include one or mutations at an amino acid position selected from the group consisting of 49, 66, 118, 119, 122, 126, 127, 131, 132, 135, 135, 138, 142, and combinations thereof relative to SEQ ID NO: 1. In some embodiments, the OMCP or variant thereof can include one or mutations at an amino acid position selected from the group consisting of 50, 67, 119, 120, 123, 127, 128, 132, 133, 136, 139, 143, and combinations thereof relative to SEQ ID NO: 2 or SEQ ID NO: 3. In some embodiments, the OMCP or variant thereof does not include any mutations at amino acid positions corresponding to 49, 66, 118, 119, 122, 126, 127, 131, 132, 135, 135, 138, 142 of SEQ ID NO: 1. In some embodiments, the OMCP or variant thereof does not include any mutations at amino acid positions corresponding to 50, 67, 119, 120, 123, 127, 128, 132, 133, 136, 139, 143 of SEQ ID NO: 2 or SEQ ID NO: 3. In some embodiments, the OMCP or variant thereof does not include any mutations at amino acid positions corresponding to 49, 66, 118, 119, 122, 126, 127, 131, 132, 135, 135, 138, 142 of SEQ ID NO: 1 except for conservative mutations, such as, by way of example, but not limitation, hydrophobic for hydrophobic amino acid mutations. In some embodiments, the OMCP or variant thereof does not include any mutations at amino acid positions corresponding to 50, 67, 119, 120, 123, 127, 128, 132, 133, 136, 139, 143 of SEQ ID NO: 2 or SEQ ID NO: 3, except for conservative mutations, such as, by way of example, but not limitation, hydrophobic for hydrophobic amino acid mutations. As discussed in the present disclosure, mutations can also be made subject to sequence identity requirements with respect to the interface regions of SEQ ID NOs: 1-3, such as 48-67 and 110-147 of SEQ ID NO: 1, 49-68 and 111-148 of SEQ ID NO: 2, or 48-66 and 111-148 of SEQ ID NO: 3.

Mutants of OMCP with reduced binding to NKG2D that have been identified are provided in Table 2 below.

TABLE 2
NKG2D Reduced Binding OMCP Mutants

| Amino | |
|---|---|
| Acid | Associated Mutations |
| D132 | D132N |
| | D132N, T31S, V68A |
| | D132G, K126N, D76V |
| K126 | K126N |
| | K126N, S71G |
| | K126N, D132G, D76V |
| K125 | K125E, F65C |
| | K125E, F92V |
| S120 | S120Y |
| | S120Y, E10A, N56K |
| D76 | D76V, D132G, K126N |
| W116 | W116R |
| | W116R, K113Q |
| R123 | R123G, D28G, F50L |
| | R123G, D21V, F128L |
| E75 | E75D |
| S71 | S71G, K126N |
| F92 | F92V, K125E |
| F65 | F65C, K125E |
| K113 | K113Q, W116R |
| E10 | E10A, N56K, S120Y |
| N56 | E10A, N56K, S120Y |
| D21 | D21V, R123G, F128L |
| F128 | D21V, R123G, F128L |
| D26 | D26G, F50L, R123G |
| F50 | D26G, F50L, R123G |
| T31 | T31S, V68A, D132N |
| V68 | T31S, V68A, D132N |
| I30 | I30, L51F, L64P, M135T |
| L51 | I30, L51F, L64P, M135T |
| L64 | I30, L51F, L64P, M135T |
| M135 | I30, L51F, L64P, M135T |
| R67 | R67S, L117P, T119N, F122L |
| L117 | R67S, L117P, T119N, F122L |
| T119 | R67S, L117P, T119N, F122L |
| F122 | R67S, L117P, T119N, F122L |

In some embodiments, the OMCP or variant thereof includes one or more of the mutations in Table 2. In some embodiments, the OMCP or variant thereof does not include any of the mutations in Table 2. It should be understood that in such embodiments, where the OMCP or variant thereof includes or does not include the mutations or any combination thereof from Table 2, these amino acid positions would correspond to SEQ ID NO: 1, while for SEQ ID NO: 2 and SEQ ID NO: 3 they would be at +1 positions. Thus, the inclusion or exclusion of such mutations should be at a corresponding position in the OMCP or variant thereof.

### Pharmaceutical Compositions

The present disclosure also provides pharmaceutical compositions. The pharmaceutical composition can include OMCP, or variant thereof, as an active ingredient and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term “parenteral” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

For topical (e.g., transdermal or transmucosal) administration, penetrants appropriate to the barrier to be permeated are generally included in the preparation. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising OMCP or a variant thereof is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of OMCP in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the compound of the invention may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying OMCP may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, OMCP may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The composition of the invention may be encapsulated in a microemulsion by any method generally known in the art.

In yet another embodiment, OMCP may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm

### Administration

In certain aspects, a pharmacologically effective amount of OMCP may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. Pheresis may be used to deliver OMCP. In certain embodiments, OMCP may be administered via an infusion (continuous or bolus).

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

For therapeutic applications, a therapeutically effective amount of OMCP is administered to a subject. A “therapeutically effective amount” is an amount of the therapeutic composition sufficient to produce a measurable response (e.g., an immunostimulatory, an anti-angiogenic response, a cytotoxic response, tumor regression, immunoinhibitory, immunosuppression, infection reduction). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, the autoimmune disease, infection, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

In some embodiments, the subject is diagnosed with and/or suffering from an autoimmune or inflammatory disease. In some embodiments, the autoimmune or inflammatory disease is selected from the group consisting of intestinal inflammation, type 1 diabetes, multiple sclerosis, rheumatoid arthritis, and chronic obstructive pulmonary disease (COPD).

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In a specific embodiment, the frequency of dosing may be twice daily.

Duration of treatment could range from a single dose administered on a one-time basis to a life-long course of therapeutic treatments. The duration of treatment can and will vary depending on the subject and the autoimmune disease or infection to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The timing of administration of the treatment relative to the disease itself and duration of treatment will be determined by the circumstances surrounding the case. Treatment could begin immediately, such as at the time of diagnosis, or treatment could begin following surgery. Treatment could begin in a hospital or clinic itself, or at a later time after discharge from the hospital or after being seen in an outpatient clinic.

It should likewise be understood that in any of the foregoing embodiments, the OMCP or variant thereof can be the only pharmaceutically active component in the composition. It should be understood that in any of the embodiments of the present disclosure, the OMCP or variant thereof can not be linked to any peptide, cytokine, antibody or fragment thereof, or any other therapeutic or targeting moiety. By way of example, but not limitation, the OMCP or variant thereof can not be linked to or conjugated to a ligand or receptor-specific antibody. However, it should be understood, that in some embodiments, the OMCP or variant thereof can be administered with another therapeutic compound. In some embodiments, the OMCP or variant thereof can be a dimer, trimer, or any other multimer of the OMCP or variant thereof. Methods of producing multimers are known to those of skill in the art.

### EXAMPLES

### Example 1—Lymphocyte Activation Assay

This example will demonstrate the capacity of OMCP to downregulate expression and/or function of NKG2D on NK and CD8 T cells but not CD4 T or T regulatory cells.

Fresh mixed splenocytes will be harvested from C57Bl/6 mice and cultured in a 12 well plate a concentration of 5×10⁶ cells per well in RPMI media supplemented with 10% fetal bovine serum and 100 IU/mL human IL2. OMCP will be added to wells for a final concentration as follows: 100 μg/mL, 50 μg/mL, 10 μg/mL, 5 μg/mL, 1 μg/mL, 0.5 μg/mL, 0.1 μg/mL, 0.05 μg/mL, 0.01 μg/mL, or 0 μg/mL. Each concentration of OMCP will be tested in three wells. Cells will then be subsequently cultured for 36 hours.

After 36 hours, cells will be evaluated via flow cytometry. Individual populations of cells will be identified using standard surface markers for NK, CD8 T, CD4 T, and CD4+Foxp3+ T regulatory cells. NKG2D expression is measured on each of these cell populations. KLRG1, CD69, and ICOS expression will be measured to evaluate relative cell activation. NKG2D function will be measured using standard killing assays such as lysis of NKG2D-responsive tumors such as Lewis Lung Carcinoma.

### Example 2—Lymphocyte Expansion Assay

The example will demonstrate the capacity of OMCP to reduce proliferation in response to IL2 on NK and CD8 T cells but not CD4 T or T regulatory cells.

Fresh mixed splenocytes will be harvested from C57Bl/6 mice and stained with CFSE dye, which binds to the DNA. Splenocytes will be subsequently cultured in a 12 well plate a concentration of 5×10⁶ cells per well in RPMI media supplemented with 10% fetal bovine serum and 1000 IU/mL human IL2 or NKG2D activation through plate-bound NKG2D crosslinking antibody. OMCP is added to wells for a final concentration as follows: 100 μg/mL, 50 μg/mL, 10 μg/mL, 5 μg/mL, 1 μg/mL, 0.5 μg/mL, 0.1 μg/mL, 0.05 μg/mL, 0.01 μg/mL, or 0 μg/mL. Each concentration of OMCP is tested in three wells. Cells will be subsequently cultured for 6 days.

After 6 days, cells will be evaluated via flow cytometry. Individual populations of cells will be identified using standard surface markers for NK, CD8 T, CD4 T, and CD4+Foxp3+ T regulatory cells. Cell proliferation will be measured by absolute cell number counts as well as CFSE fluorescence. CFSE dye binds to DNA and fluorescence is reduced with proliferation in a predictable Log 2 manner.

### Example 3—In Vivo Dosing with OMCP

This example will demonstrate the capacity of OMCP to downregulate expression and function of NKG2D on NK and CD8 T cells, and whether or not OMCP avoids the downregulation of CD4 T or T regulatory cells. If OMCP inhibits NKG2D expression, the doses assayed will be used for dose selection in subsequent assays.

Mice will be injected with OMCP via subcutaneous dosing 1× daily for 5 days. OMCP will be dosed as follows: 50 mg/kg, 25 mg/kg, 10 mg/kg, 5 mg/kg, 2.5 mg/kg, and 1 mg/kg, or saline control. On day 6, one day after the last dose, mice will be euthanized and the splenocytes harvested for population analysis via flow cytometry. Individual populations of cells will be identified using standard surface markers for NK, CD8 T, CD4 T, and CD4+Foxp3+ T regulatory cells. NKG2D expression and function is measured on each of these cell populations. KLRG1, CD69, and ICOS expression will be measured to evaluate relative cell activation.

### Example 4—In Vivo Dosing with OMCP

The ability of OMCP to inhibit cytotoxic lymphocyte functions will be evaluated using an in vitro killing assay after in vivo treatment as a measure of inhibited functionality. Mice will be injected with OMCP or saline control via subcutaneous dosing 1× daily for 5 days. OMCP will be dosed at the concentration identified in Example 3 to optimally inhibit NKG2D expression. On day 6, one day after the last dose, mice will be euthanized and the splenocytes harvested.

Target cells (LM2 lung cancer, YAC-1 lymphoma, and LLC lung cancer lines) will be pre-treated with chromium and seeded into 96 well plates. Bulk splenocytes collected from the treated mice will be seeded into the same plates at varying concentrations to achieve an effector to target ratio as follows: 0, 15.6:1, 31.25:1, 62.5:1, 125:1, 250:1, 500:1. Cells will be incubated for 4 hours at 37° C. prior to measuring chromium release from the target cells. Chromium release is indicative of killing function.

### Example 5—Rheumatoid Arthritis Model

This example will demonstrate the capacity of OMCP to; (1) reduce body weight due to fluid accumulation in immunized mice; (2) reduce paw volume; (3) reduce bone marrow density; and (4) reduce serum inflammation. The effects on these 4 measurements will indicate the potential efficacy of OMCP in the treatment of rheumatoid arthritis.

Collagen induced arthritis (CIA) will be induced in mice using incomplete Freund's adjuvant (IFA) in combination with type II collagen according to previously reported methodology. A cohort of naïve mice will be maintained as normal controls. At 9 days post-immunization, immunized mice will be injected with OMCP or saline control via subcutaneous dosing 1× daily for 5 days. OMCP will be dosed at the concentration identified in Example 3 to optimally inhibit NKG2D expression. Body weight and paw volume will be monitored pre- and post-immunization and throughout treatment.

On day 15, one day after the last dose OMCP, mice will be euthanized. Serum will be evaluated for inflammatory cytokine concentrations, NK and CD8 T cells profiled for activation markers, and bone marrow density evaluated (BMD).

### Example 6—Amelioration of COPD

Chronic obstructive pulmonary disease (COPD) is characterized by peribronchial and perivascular inflammation and largely irreversible airflow obstruction. This example will demonstrate the capacity of OMCP to ameliorate the development or progression of COPD by evaluating the following: (1) histopathological measures of infiltration of cells into the parenchyma; (2) mucosal secretion (3) ticking of airway epithelium; (4) alveolar enlargement; (5) airway remodeling; (6) goblet cell hyperplasia; (7) numbers of infiltrating lymphocytes, NK cells, CD8 T cells, and macrophages, and the level of activation of these cells, as measured via flow cytometry; and (8) blood serum markers of chronic inflammation such as TNFα, IL8, and IL10.

COPD will be induced in a cohort of mice via whole-body exposure to cigarette smoke at 150 mg total particulate matter per cubic meter for 4 hours per day, 5 days per week, for 6 months. Throughout this period, mice will be injected with OMCP or saline control via subcutaneous dosing 1× daily for 5 days per week. OMCP will be dosed at the concentration identified in Example 3 to optimally inhibit NKG2D expression. Mice will be euthanized at the end of this period, blood serum collected, and the lungs collected for evaluation. One half of the lung will be preserved for histopathological analysis, and the second half will be digested and profiled for infiltrating lymphocytes. Using standard markers, total lymphocyte counts, NK cells, CD8 T cells, and macrophage populations will be evaluated. NK and CD8 T cells will be evaluated for expression of the granulocyte marker CD107a. Blood serum will profiled for TNFα, IL8, and IL10 expression.

### Example 7—Assessment of Binding Affinity

Binding affinity of OMCP and variants thereof will be assessed by surface plasmon resonance. A ProteOn XPR36 instrument (Bio-Rad) will used to determine the kinetics of protein:protein interactions. All experiments will be carried out at a flow rate of 100 μl/min, 25° C., and in running buffer containing 1×PBS, pH 7.4, 0.005% Tween 20. GLC chips will be activated with 1-Ethyl-3-(3-dimethylaminopropyl) carbodiimide (EDC)/N-Hydroxysuccinimide (NHS) for amine coupling of proteins. On one chip ˜1000 RUs of human NKG2D will be coupled. ˜500 RUs of each OMCP or variant thereof will be coupled to a second chip. Ethanolamine will then be used to quench unreacted esters.

OMCP or variant thereof binding to human NKG2D will be determined over a suitable range. Human NKG2D binding will be regenerated with pulses of 10 mM HCl. Data will be analyzed using ProteOn analysis software with OMCP or variant thereof:NKG2D curves fitted using a 1:1 langmuir binding model.

1. A method for treating an autoimmune or inflammatory disease in a subject diagnosed with or suffering from the autoimmune or inflammatory disease, comprising administering to the subject a therapeutically effective amount of a composition, wherein the composition comprises Orthopoxvirus Major Histocompatibility complex (MHC) class I-like protein (OMCP) or a variant thereof.

2. The method of claim 1, wherein the OMCP or variant thereof comprises an amino acid sequence that has at least 80% sequence identity to any one of SEQ ID NOs: 1-3.

3-7. (canceled)

8. The method of claim 1, wherein the OMCP or variant thereof comprises the amino acid sequence of SEQ ID NO: 1, SEQ ID NO: 2, or SEQ ID NO: 3.

9.-10. (canceled)

11. The method of claim 1, wherein the OMCP or variant thereof has a binding affinity for human NKG2D of from about 0.1 nM to about 1000 nM.

12.-13. (canceled)

14. The method of claim 1, wherein the OMCP or variant thereof is not linked to a cytokine.

15.-17. (canceled)

18. The method of claim 1, wherein the OMCP or variant thereof further comprises polyethylene glycol (PEG), and wherein the PEG is selected from the group consisting of PEG-10K, PEG-20K and PEG-40K.

19.-33. (canceled)

34. The method of claim 1, wherein the OMCP or variant thereof comprises N-linked glycans.

35.-58. (canceled)

59. The method of claim 1, wherein the OMCP or variant thereof is linked to a human immunoglobin IgG Fc chain.

60.-107. (canceled)

108. The method of claim 1, wherein the OMCP or variant thereof is present in the composition in a form selected from the group consisting of encapsulated in a liposome or micelle, in a miroemulsion, or linked to a dendrimer.

109.-205. (canceled)

206. The method of claim 1, wherein the composition further comprises an excipient, and wherein the excipient is selected from the group consisting of a diluent, a binder, a filler, a buffering agent, a pH modifying agent, a disintegrant, a dispersant, a preservative, a lubricant, taste-masking agent, a flavoring agent, or a coloring agent.

207. (canceled)

208. The method of claim 206, wherein the excipient is present in the composition in an amount from about 1% to about 99% by weight.

209. The method of claim 1, wherein the step of administering is by parenteral administration, topical administration, injection, or oral administration.

210.-221. (canceled)

222. The method of claim 1, wherein the autoimmune or inflammatory disease is selected from the group consisting of intestinal inflammation, type I diabetes, multiple sclerosis, rheumatoid arthritis, and chronic obstructive pulmonary disease (COPD).

223. The method of claim 1, wherein the OMCP or variant thereof is the only pharmaceutically active component in the composition.

224. The method of claim 1, wherein the OMCP or variant thereof is not linked to or conjugated to any other peptide.

225. The method of claim 1, wherein the OMCP or variant thereof is not linked to or conjugated to an antibody or fragment thereof.

226. The method of claim 1, wherein the OMCP or variant thereof is not linked to or conjugated to any other therapeutic moiety.

227. The method of claim 1, wherein the OMCP or variant thereof is not linked to or conjugated to a targeting moiety.

228. The method of claim 1, wherein the composition further comprises another therapeutic compound.


SEQUENCE LISTING (4 sequences)

SEQ ID NO: 1 - protein, 152 aa, Cowpox virus
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTG

SEQ ID NO: 2 - protein, 150 aa, Cowpox virus
GHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKR

SEQ ID NO: 3 - protein, 150 aa, Monkeypox virus
HKLVHYFNLKINGSDITNTADILLDNYPIMTFDGKDIYPSIAFMVGNKLFLDLYKNIFVEFFRLFRVSVSSQYEELEYYYSCDYTNNRPTIKQHYFYNGEEYTEIDRSKKATNKNSWLITSGFRLQKWFDSEDCIIYLRSLVRRMEDSNK

SEQ ID NO: 4 - protein, 171 aa, Cowpox virus
MHLQYYAISLVSLVSLIDCHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTG